(19) United States
(12) Patent Application Publication
(10) Pub. No.: US 2026/0347279 A1
(43) Pub. Date: Nov. 5, 2026

(54) METHOD FOR PRODUCING CARBONYL COMPOUND

(71) Applicant: FUJIFILM Corporation, Tokyo (JP)

(72) Inventors: Ryo NISHIO, Kanagawa (JP); Yuki YAMADA, Kanagawa (JP); Kenji WADA, Kanagawa (JP)

(73) Assignee: FUJIFILM Corporation, Tokyo (JP)

(21) Appl. No.: 19/697,539

(22) Filed: Jun. 3, 2026

Related U.S. Application Data
(63) Continuation of application No. PCT/JP2025/004086, filed on Feb. 7, 2025.

(30) Foreign Application Priority Data
Feb. 9, 2024 (JP) 2024-018711

(51) Int. Cl. C07B 41/06

(52) U.S. Cl. CPC C07B 41/06

(57) ABSTRACT

A method for producing a carbonyl compound, the method comprising: reacting an active hydrogen-containing compound having, in a molecule, an active hydrogen-containing group formed by bonding a hydrogen atom to an atom selected from an oxygen atom, a nitrogen atom, and a sulfur atom, with a carbonate diester compound in a presence of a quaternary ammonium salt represented by General Formula (1) or (2), thereby introducing a carbonyl group into the active hydrogen-containing compound,

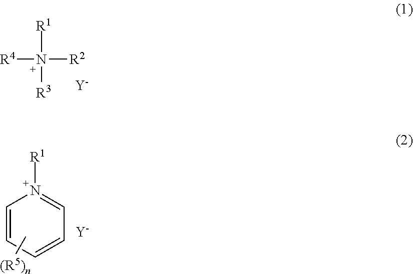

in General Formulae (1) and (2), R¹ represents an organic group having a formula weight of 65 or more, R² to R⁴ each represent an alkyl group, and at least two of R², R³, or R⁴ are alkyl groups having 6 or fewer carbon atoms, R⁵ represents an alkyl group having 1 to 20 carbon atoms, and n is an integer of 0 to 2, and Y⁻ represents a counterion.

### CROSS-REFERENCE TO RELATED APPLICATIONS

This application is a Continuation of PCT International Application No. PCT/JP2025/004086 filed on Feb. 7, 2025, which claims priority under 35 U.S.C. § 119(a) to Japanese Patent Application No. 2024-018711 filed in Japan on Feb. 9, 2024. Each of the above applications is hereby expressly incorporated by reference, in its entirety, into the present application.

### BACKGROUND OF THE INVENTION

### 1. Field of the Invention

The present invention relates to a method for producing a carbonyl compound.

### 2. Description of the Related Art

A halogenated carbonyl compound such as phosgene is known as a reagent for introducing a carbonyl group into various compounds containing active hydrogen. For example, an isocyanate compound, a urea compound, or the like can be obtained by a reaction between phosgene and a primary amine. In addition, a carbonate compound, a chloroformate compound, or the like can be obtained by a reaction between phosgene and an alcohol. Furthermore, a chlorothioformate or the like can also be obtained by a reaction between phosgene and a thiol.

However, the halogenated carbonyl compound has high toxicity, and many of the halogenated carbonyl compounds are in a gaseous state at room temperature, and thus handling requires great care.

In order to introduce, without using a halogenated carbonyl compound, a carbonyl group into an active hydrogen-containing compound, a method of reacting a carbonate diester compound such as diphenyl carbonate, which has a highly electron-withdrawing leaving group, with an active hydrogen-containing compound is also known. In addition, it has also been studied to use a dialkyl carbonate having a lower alkyl group instead of diphenyl carbonate or the like. For example, JP2012-106982A describes that an ester exchange reaction between dimethyl carbonate and 1-adamantanol, which is a tertiary alcohol, is carried out in the presence of a quaternary ammonium salt having a specific structure ([Me(n-C₁₂H₂₅)₃N]⁺[OCO₂Me]⁻, Me: methyl). In the method described in JP2012-106982A, since methanol, which is a by-product, has a low boiling point, it is easy to remove methanol from the reactor system.

### SUMMARY OF THE INVENTION

An object of the present invention is to provide a method for producing a carbonyl compound, in which a carbonyl group can be introduced into an active hydrogen-containing compound with higher efficiency by a reaction between a carbonate diester compound and an active hydrogen-containing compound, and as a result, a target carbonyl compound can be obtained with high efficiency.

The present inventors have conducted repeated studies to introduce a carbonyl group into an active hydrogen-containing compound with high efficiency by reacting a carbonate diester compound with an active hydrogen-containing compound. As a result, it has been found that the catalytic activity of the quaternary ammonium salt described in JP2012-106982A is not sufficient, and the introduction efficiency of the carbonyl group into the active hydrogen-containing compound is significantly increased by setting four substituents of the quaternary ammonium salt to a specific combination. The present invention has been further studied based on these findings, and has been completed.

The above-described objects of the present invention have been achieved by the following methods.

[1]

A method for producing a carbonyl compound, the method including: reacting an active hydrogen-containing compound having, in a molecule, an active hydrogen-containing group formed by bonding a hydrogen atom to an atom selected from an oxygen atom, a nitrogen atom, and a sulfur atom, with a carbonate diester compound in a presence of a quaternary ammonium salt represented by General Formula (1) or (2), thereby introducing a carbonyl group into the active hydrogen-containing compound,

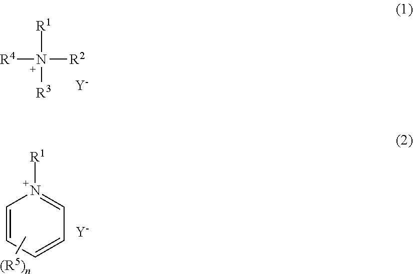

in General Formulae (1) and (2), R¹ represents an organic group having a formula weight of 65 or more, R² to R⁴ each represent an alkyl group, and at least two of R², R³, or R⁴ are alkyl groups having 6 or fewer carbon atoms, R⁵ represents an alkyl group having 1 to 20 carbon atoms, and n is an integer of 0 to 2, and Y⁻ represents a counterion.

[2]

The method for producing a carbonyl compound according to [1], in which in General Formulae (1) and (2), R¹ represents an alkyl group having 5 to 30 carbon atoms or a polymer structure.

[3]

The method for producing a carbonyl compound according to [1] or [2], in which in General Formulae (1) and (2), R¹ is a polymer structure having a crosslinking structure.

[4]

The method for producing a carbonyl compound according to any one of [1] to [3], in which the quaternary ammonium salt is present as a solid in a reactor system of the reaction.

[5]

The method for producing a carbonyl compound according to any one of [1] to [4], in which the reaction is performed in a flow reactor system.

[6]

The method for producing a carbonyl compound according to [5], the method further including: causing a liquid containing the active hydrogen-containing compound and the carbonate diester compound to flow through an inside of a column filled with the quaternary ammonium salt.

[7]

The method for producing a carbonyl compound according to any one of [1] to [6], in which in General Formula (1), at least two of R², R³, or R⁴ are methyl.

[8]

The method for producing a carbonyl compound according to any one of [1] to [7], in which in General Formulae (1) and (2), Y⁻ is selected from a halide anion, a sulfonate anion, a phosphate anion, a phosphonate anion, a perchlorate anion, and a fluorophosphate anion.

[9]

The method for producing a carbonyl compound according to any one of [1] to [8], in which the carbonate diester compound is a dialkyl carbonate compound.

[10]

The method for producing a carbonyl compound according to [9], in which the carbonate diester compound is dimethyl carbonate.

[11]

The method for producing a carbonyl compound according to any one of [1] to [10], in which the active hydrogen-containing compound is selected from a polyhydric alcohol compound, a polyvalent amine compound, and a polyvalent thiol compound.

[12]

The method for producing a carbonyl compound according to any one of [1] to [11], in which the carbonyl compound obtained by the production method is a monomer and/or a multimer.

[13]

The method for producing a carbonyl compound according to any one of [1] to [12], the method further including: irradiating a reactor system of the reaction with a microwave to heat the reactor system.

In the present invention, a numerical range expressed using “to” means a range including numerical values described before and after “to” as a lower limit value and an upper limit value.

In the present invention, a group in which substitution or unsubstitution is not specified includes a group having any substituent as long as the intended effect is not impaired. For example, “alkyl group” has a meaning including a substituted alkyl group and an unsubstituted alkyl group. In addition, for example, “phenyl” has a meaning including a substituted phenyl and an unsubstituted phenyl.

According to the method for producing a carbonyl compound according to the embodiment of the present invention, a carbonyl group can be introduced into an active hydrogen-containing compound with higher efficiency by the reaction between a carbonate diester compound and an active hydrogen-containing compound, and as a result, a target carbonyl compound can be obtained with high efficiency.

### BRIEF DESCRIPTION OF THE DRAWINGS

FIG. 1 is a view schematically showing an example of a flow reaction system.

### DESCRIPTION OF THE PREFERRED EMBODIMENTS

### [Method for Producing Carbonyl Compound]

The method for producing a carbonyl compound according to the embodiment of the present invention (hereinafter, referred to as “production method according to the embodiment of the present invention”) includes reacting a compound (active hydrogen-containing compound) having, in a molecule, an active hydrogen-containing group in which a hydrogen atom is bonded to an atom selected from an oxygen atom, a nitrogen atom, and a sulfur atom, with a carbonate diester compound in the presence of a quaternary ammonium salt having a specific structure, thereby introducing a carbonyl group into the active hydrogen-containing compound. The reaction itself of reacting an active hydrogen-containing compound with a carbonate diester compound, thereby introducing a carbonyl group into the active hydrogen-containing compound is known. The present invention is characterized in that a quaternary ammonium salt having a specific structure described later, which has not been known to have catalytic activity for this reaction, is used. The production method according to the embodiment of the present invention will be described in detail.

### <Active Hydrogen-Containing Compound>

The active hydrogen-containing compound is not particularly limited as long as it is a compound having active hydrogen in the molecule. For example, a compound having at least one group selected from —OH, —COOH, —NH₂, —NHR (R is a substituent), and —SH can be widely used. The active hydrogen-containing compound is, for example, at least one compound of a primary amine compound, a secondary amine compound, a hydroxy compound (the term “hydroxy compound” is used to mean a compound including both a compound having an alcoholic hydroxyl group and a compound having a phenolic hydroxyl group), a thiol compound, a carboxylic acid compound, or an amino acid compound, and among these, at least one of a primary amine compound, a secondary amine compound, a hydroxy compound, or a thiol compound is preferable.

The active hydrogen-containing compound preferably has a molecular weight of 40 to 1,000 and more preferably 60 to 500.

In a case where the carbonyl compound obtained by the production method according to the embodiment of the present invention is used as a monomer or a prepolymer (in a case of being used as a raw material of a polymer), the active hydrogen-containing compound can be selected from a polyvalent hydroxy compound (a compound having two or more hydroxy groups), a polyvalent amine compound (a compound having two or more amino groups (preferably, unsubstituted amino groups)), and a polyvalent thiol compound (a compound having two or more thiol groups), and among these, it is preferable to select the active hydrogen-containing compound from a diol compound (two hydroxy groups of the diol compound may be alcoholic or phenolic), a diamine compound, or a dithiol compound. For example, in a case where the carbonyl compound obtained by the production method according to the embodiment of the present invention is used as a monomer or a prepolymer for obtaining a polycarbonate compound, a polyvalent hydroxy compound can be used as the active hydrogen-containing compound, and a diol compound is preferably used. In addition, for example, in a case where the carbonyl compound obtained by the production method according to the embodiment of the present invention is used as a monomer or a prepolymer for obtaining a polyurea compound, a polyvalent amine compound can be used as the active hydrogen-containing compound, and a diamine compound is more preferably used. In addition, in a case where the carbonyl compound is used as a monomer or a prepolymer for obtaining a polythioamide compound, polyvalent thiol can be used as the active hydrogen-containing compound, and a dithiol compound is more preferably used. In addition, a plurality of kinds of the active hydrogen-containing compounds (for example, a diol compound and a diamine compound) may be used, or a plurality of kinds of active hydrogens (for example, a hydroxy group and an amino group) may be included in the same compound.

### <Carbonate Diester Compound>

The carbonate diester compound is a compound represented by R^{a}O—C(═O)—OR^{b}. R^{a} and R^{b} each represent a substituent, and are preferably an alkyl group or an aryl group. R^{a} and R^{b} may be the same or different from each other, and are preferably the same.

The alkyl group that can be adopted as R^{a} and R^{b} has preferably 1 to 10 carbon atoms, more preferably 1 to 8 carbon atoms, still more preferably 1 to 6 carbon atoms, even more preferably 1 to 4 carbon atoms, and even still more preferably 1 to 3 carbon atoms. In a case where the alkyl group that can be adopted as R^{a} and R^{b} has 3 or more carbon atoms, the alkyl group may be linear or branched. The alkyl group that can be adopted as R^{a} and R^{b} is still more preferably methyl or ethyl, and is even still more preferably methyl.

The aryl group that can be adopted as R^{a} and R^{b} is preferably phenyl or naphthyl, and is still more preferably phenyl.

The alkyl group or aryl group (preferably phenyl or naphthyl) that can be adopted as R^{a} and R^{b} may have a substituent as long as the effect of the present invention is not impaired. The alkyl group or aryl group that can be adopted as R^{a} and R^{b} is preferably an unsubstituted alkyl group or an unsubstituted aryl group.

The carbonate diester compound is preferably a dialkyl carbonate compound or a diphenyl carbonate compound. From the viewpoint that by-products can be removed with high efficiency while carrying out the reaction with the active hydrogen-containing compound at a lower temperature, a dialkyl carbonate compound having an alkyl group having 1 to 4 carbon atoms is preferable, diethyl carbonate (which generates ethanol having a low boiling point as a by-product) or dimethyl carbonate (which generates methanol having a low boiling point as a by-product) is more preferable, and dimethyl carbonate is still more preferable. In addition, in a case where a dialkyl carbonate compound is used, diethyl carbonate or dimethyl carbonate is preferable, and dimethyl carbonate is more preferable from the viewpoint of the introduction efficiency of the carbonyl group.

### <Quaternary Ammonium Salt>

In the production method according to the embodiment of the present invention, the active hydrogen-containing compound and the carbonate diester compound are reacted with each other using a quaternary ammonium salt represented by General Formula (1) or (2) as a reaction catalyst.

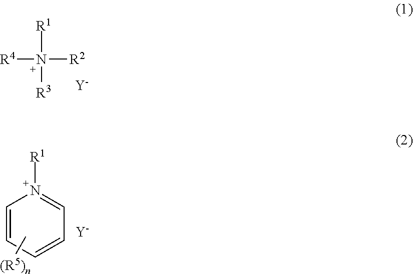

In General Formulae (1) and (2), R¹ represents an organic group having a formula weight of 65 or more. R² to R⁴ each represent an alkyl group, and at least two of R², R³, or R⁴ are alkyl groups having 6 or fewer carbon atoms, R⁵ represents an alkyl group having 1 to 20 carbon atoms, and n is an integer of 0 to 2. Y⁻ represents a counterion.

The organic group having a formula weight of 65 or more as R¹ is preferably an alkyl group, an aromatic group, or a polymer structure. The organic group having a formula weight of 65 or more as R¹ is preferably an organic group having a formula weight of 70 or more.

The alkyl group that can be adopted as R¹ is not particularly limited as long as the formula weight thereof is 65 or more. Therefore, the alkyl group that can be adopted as R¹ is an alkyl group having 5 or more carbon atoms, and it is preferably an alkyl group having 5 to 30 carbon atoms, more preferably an alkyl group having 6 to 28 carbon atoms, still more preferably an alkyl group having 8 to 26 carbon atoms, and particularly preferably an alkyl group having 10 to 24 carbon atoms. The alkyl group that can be adopted as R¹ may be linear or branched. In a case where the alkyl group that can be adopted as R¹ is branched, the number of branches is preferably 1 (one branched structure) or 2 (two branched structures), and more preferably 1 (one branched structure). The alkyl group that can be adopted as R¹ may have a substituent as long as the effect of the present invention is not impaired. In this case, in a case where the formula weight of the entire alkyl group including the substituent is 65 or more, the alkyl group corresponds to the “organic group having a formula weight of 65 or more” described above. The alkyl group that can be adopted as R¹ is preferably an unsubstituted alkyl group or an alkyl group having an aryl group (preferably phenyl) as a substituent, and more preferably an unsubstituted alkyl group.

The aromatic group that can be adopted as R¹ is an aryl group or a heteroaryl group, and an aryl group is preferable. The aromatic group may be a monocyclic ring or a fused ring, and a monocyclic ring is preferable. The number of ring members in the ring of the aromatic group (the number of ring members in each monocyclic ring constituting the fused ring in a case of a fused ring) is preferably 5 or 6. The aromatic group is more preferably phenyl. The aromatic group that can be adopted as R¹ may have a substituent as long as the effect of the present invention is not impaired.

In a case of the quaternary ammonium salt represented by General Formula (2), R¹ is preferably an alkyl group having a formula weight of 65 or more or a polymer structure.

The polymer structure that can be adopted as R¹ is not particularly limited as long as it is a polymer structure. The polymer structure may be a step-growth polymerization structure or an addition polymerization structure, and is preferably an addition polymerization structure of a monomer having a carbon-carbon double bond. For example, an addition polymerization structure (polystyrene compound structure) containing a styrene compound as a monomer component is preferable as the polymer structure that can be adopted as R¹. The polymer structure that can be adopted as R¹ is preferably a hydrocarbon structure (hydrocarbon polymer structure). In addition, the polymer structure that can be adopted as R¹ preferably has a crosslinking structure. By introducing the crosslinking structure, in the production method according to the embodiment of the present invention, it is also preferable that the quaternary ammonium salt is present in the reactor system in a solid form and the active hydrogen-containing compound and the carbonate diester compound are reacted with each other. In this case, the polymer structure also functions as a carrier that supports a catalyst.

In a case where R¹ in General Formula (1) or (2) is a polymer structure, it is preferable that the polymer structure also has one or two or more structural moieties corresponding to the quaternary ammonium salt represented by General Formula (1) or (2). That is, it is preferable that a plurality of the quaternary ammonium salts represented by General Formula (1) or (2) share one polymer structure as each R¹. For example, in the quaternary ammonium salt “A-11” described in Examples later, the polymer structure corresponding to R¹ is a crosslinked polystyrene compound structure, and the crosslinked polystyrene compound structure has a plurality of structural moieties obtained by removing R¹ from General Formula (1). Such a structure is also included in the quaternary ammonium salt represented by General Formula (1).

In a case where R¹ is a polymer structure, a bond between the polymer structure as R¹ and the N atom in General Formula (1) or (2) may be through a linking group. In this case, the linking group is positioned as a structural moiety of R¹. For example, the quaternary ammonium salt “A-11” described in Examples later can be interpreted to mean that —N⁺(Me)₃ is bonded to a polystyrene compound structure through methylene, and the methylene is interpreted as a group included in the polystyrene compound structure. However, in the quaternary ammonium salt “A-11”, —N⁺(Me)₃ can also be interpreted as being directly bonded to p-methylstyrene, which is a styrene compound, and being subjected to addition polymerization, and under this interpretation, the polymer structure as R¹ and —N⁺(Me)₃ are directly bonded to each other. In any case of the interpretation, it is clear that the quaternary ammonium salt “A-11” is included in General Formula (1), and thus the technical scope of the present invention is objectively clear.

The molecular weight of the polymer structure that can be adopted as R¹ is not particularly limited and is appropriately set. For example, the weight-average molecular weight can be set to 1,000 to 1,000,000.

R² to R⁴ in General Formula (1) represent an alkyl group as described above, and at least two of R², R³, or R⁴ are alkyl groups having 6 or fewer carbon atoms. It is also preferable that all of R² to R⁴ are alkyl groups having 6 or fewer carbon atoms. The alkyl group having 6 or fewer carbon atoms that can be adopted as R² to R⁴ preferably has 5 or fewer carbon atoms, more preferably has 4 or fewer carbon atoms, still more preferably has 3 or fewer carbon atoms, and is preferably ethyl or methyl and particularly preferably methyl. Therefore, it is preferable that at least two of R² to R⁴ are methyl.

In a case where one of R² to R⁴ is an alkyl group having 7 or more carbon atoms, the number of carbon atoms in the alkyl group is preferably 7 to 30, more preferably 8 to 22, and still more preferably 10 to 22.

In a case where the alkyl group in R² to R⁴ has 3 or more carbon atoms, the alkyl group may be linear or branched, and a linear alkyl group is preferable. In a case of having a branch, one branched structure is preferable. The alkyl group in R² to R⁴ may have a substituent as long as the effect of the present invention is not impaired, but an unsubstituted alkyl group is preferable. In a case where the alkyl group in R² to R⁴ has a substituent, the above-described number of carbon atoms is the total number of carbon atoms including the substituent.

R⁵ in General Formula (2) represents an alkyl group having 1 to 20 carbon atoms as described above. R⁵ preferably has 1 to 18 carbon atoms, more preferably has 1 to 16 carbon atoms, still more preferably has 1 to 14 carbon atoms, even still more preferably has 1 to 12 carbon atoms, even further still more preferably has 1 to 10 carbon atoms, even further still more preferably has 1 to 8 carbon atoms, and particularly preferably has 1 to 6 carbon atoms. n representing the number of R's is 0 to 2 as described above, and is preferably 0 or 1. In a case where n is 1 or 2, R⁵ is preferably bonded to a meta position or a para position with respect to the N atom constituting the pyridinium ring. In addition, in a case where n representing the number of R⁵'s is 1, R⁵ is preferably bonded to a para position with respect to the N atom constituting the pyridinium ring. The alkyl group having 1 to 20 carbon atoms in R⁵ may be linear or branched. In addition, the alkyl group having 1 to 20 carbon atoms in R⁵ may have a substituent as long as the effect of the present invention is not impaired, but an unsubstituted alkyl group is preferable. In a case where the alkyl group in R⁵ has a substituent, the above-described number of carbon atoms is the total number of carbon atoms including the substituent.

It is preferable that each group that can be adopted as R¹ to R⁵ has a hydrocarbon structure as a whole.

The counterion (anion) that can be adopted as Y⁻ is not particularly limited. Examples thereof include a counterion selected from a halide anion, a sulfonate anion, a phosphate anion, a phosphonate anion, a perchlorate anion, and a fluorophosphate anion (preferably a tetrafluorophosphate anion or a hexafluorophosphate anion). The counterion that can be adopted as Y⁻ is preferably a halide anion. The halide anion is preferably a bromide ion or a chloride ion.

Preferred specific examples of the quaternary ammonium salt represented by General Formula (1) or (2) are shown below. Me represents methyl, Bu represents n-butyl, and OTs represents a tosylate anion.

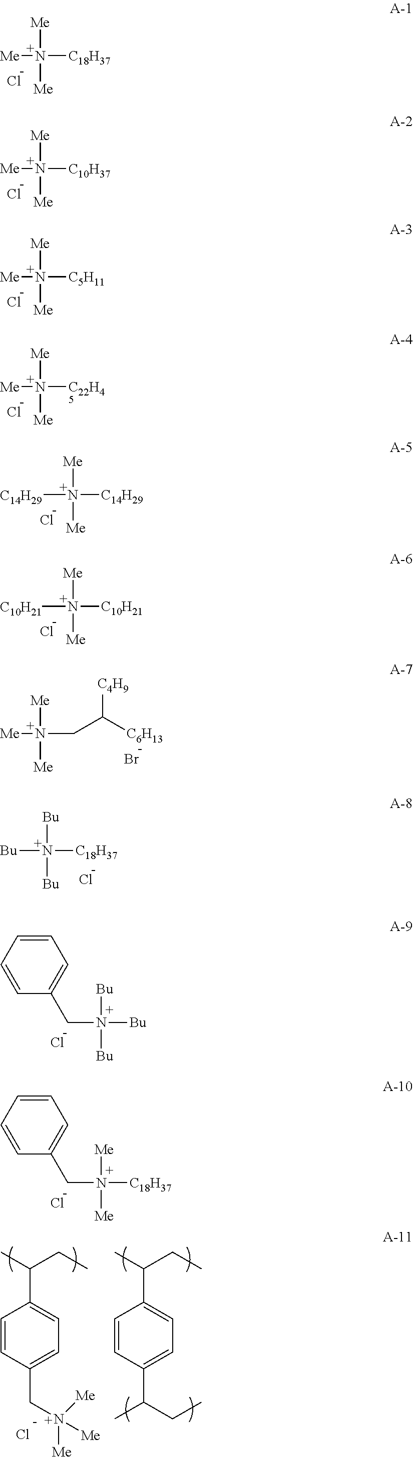

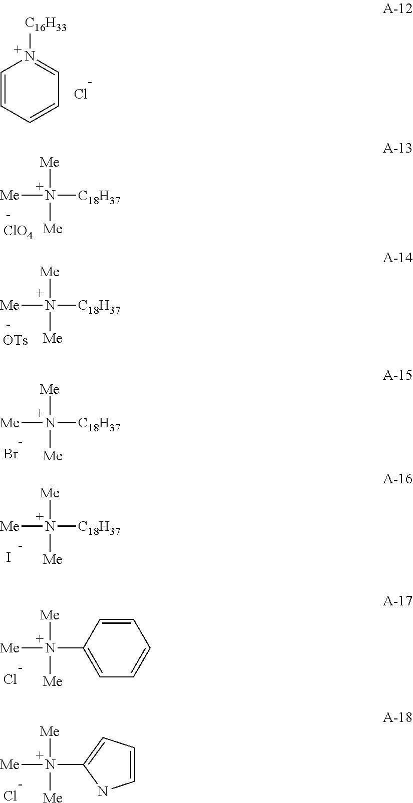

### <Carbonyl Group Introduction Reaction>

In the production method according to the embodiment of the present invention, the active hydrogen-containing compound and the carbonate diester compound are reacted with each other using the quaternary ammonium salt having a unique structure represented by General Formula (1) or (2) as a reaction catalyst (this reaction is referred to as “the reaction of the present invention”). In a case where the above-described quaternary ammonium salt is applied, the efficiency of introducing the carbonyl compound into the active hydrogen-containing compound can be further increased in a case where diphenyl carbonate having an electron withdrawing leaving group is used as a source of the carbonyl group, and the efficiency of introducing the carbonyl group into the active hydrogen-containing compound can be sufficiently increased even in a case where dialkyl carbonate having an electron withdrawing leaving group is used. The reason for this is not clear, but it is considered that one of the reasons is that the quaternary ammonium salt has a structure represented by General Formula (1) or (2), and the solubility or the affinity with the raw material is increased at another site while the steric hindrance is reduced at a specific site, and the catalytic action is made more efficient.

The reaction itself of reacting the active hydrogen-containing compound with the carbonate diester compound to introduce the carbonyl group into the active hydrogen-containing compound is well known, and as an example of the reaction of the carbonate diester compound with the monoalcohol compound, two examples of the reaction formula are as follows. R^{A}O—C(═O)—OR^{A} can be, for example, dimethyl carbonate or diphenyl carbonate (R^{A}: methyl or phenyl), and R^{B}—OH is, for example, 1-decanol (R^{B}: decyl).

R^{A}O—C(═O)—OR^{A}+R^{B}—OH

R^{A}O—C(═O)—OR^{B}+R^{A}—OH

R^{A}O—C(═O)—OR^{A}+2R^{B}—OH

R^{B}O—C(═O)—OR^{B}+2R^{A}—OH

Although two reaction formulae are shown above, both “R^{A}O—C(═O)—OR^{B}” in the first formula and “R^{B}O—C(═O)—OR^{B}” in the second formula can be positioned as the target carbonyl compound in the present invention. In addition, since the generation of the target carbonyl compound is an equilibrium reaction with the raw material, the generation ratio of “R^{A}O—C(═O)—OR^{B}” and “R^{B}O—C(═O)—OR^{B}” as a mixture can be controlled by controlling the amount ratio of “R^{A}O—C(═O)—OR^{A}” and “R^{B}—OH” which are raw materials, the reaction time, the reaction temperature, the concentration, and the like.

In the present invention, by using the quaternary ammonium salt represented by General Formula (1) or (2) as a reaction catalyst, the generation efficiency of the target carbonyl compound can be significantly increased.

As an example of a reaction different from the above, three examples of a reaction formula of a carbonate diester compound and a diol compound are shown below. R^{A} has the same meaning as described above, and HO-L^{B}-OH is, for example, 1,4-butanediol (L^{B}: butylene).

R^{A}O—C(═O)—OR^{A}+HO-L^{B}-OH

R^{A}O—C(═O)—O-L^{B}-OH+R^{A}—OH

2R^{A}O—C(═O)—OR^{A}+HO-L^{B}-OH

R^{A}O—C(═O)—O-L^{B}-O—C(═O)—OR^{A}+2R^{A}—OH

R^{A}O—C(═O)—OR^{A}+2HO-L^{B}-OH

HO-L^{B}-O—C(═O)—O-L^{B}-OH+2R^{A}—OH

Although three reaction formulae are shown above, all of “R^{A}O—C(═O)—O-L^{B}-OH” in the first formula, “R^{A}O—C(═O)—O-L^{B}-O—C(═O)—OR^{A}” in the second formula, and “HO-L^{B}-O—C(═O)—O-L^{B}-OH” in the third formula can be positioned as the target carbonyl compound (for example, a monomer for obtaining polycarbonate) in the present invention. In addition, the composition of the reaction product can also be controlled by controlling the amount ratio of “R^{A}O—C(═O)—OR^{A}” and “HO-L^{B}-OH” as raw materials, the reaction time, the reaction temperature, the concentration, and the like.

In addition, by using the quaternary ammonium salt represented by General Formula (1) or (2) as a reaction catalyst, the production efficiency of the target carbonyl compound can be significantly increased.

The reaction of the present invention is an equilibrium reaction, and for example, by reacting the carbonate diester compound in an excessive amount, the equilibrium of each of the above reaction formulae can be moved to the right side, and the target carbonyl compound can be more efficiently produced.

Regarding the reaction between the carbonate diester compound and the diol compound, the type of the product actually obtained is not limited to the product shown in the above reaction formula. For example, in a case where dimethyl carbonate is used as the carbonate diester compound and the diol compound is C-2 used in Examples described later, the following reaction product can be generated by the reaction of the present invention, including unreacted substances. All of the reaction products other than the unreacted diol compound are positioned as the target carbonyl compound in the present invention. In addition, by adjusting the reaction conditions, the following reaction product can be further polymerized and oligomerized or polymerized, and the oligomer or polymer can be contained in the reaction product.

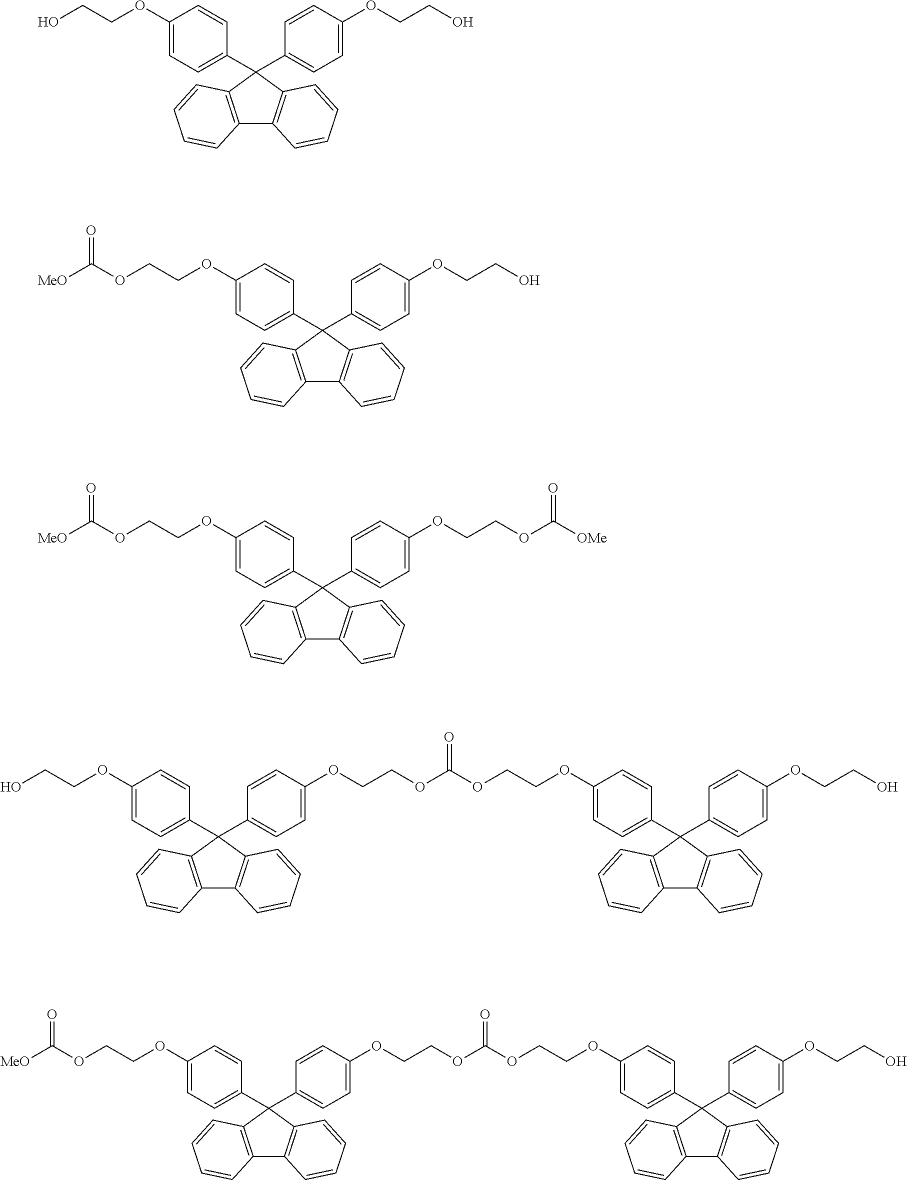 [SMILES: OCCOc1ccc(C2(c3ccc(OCCO)cc3)c3ccccc3-c3ccccc32)cc1 ; COC(=O)OCCOc1ccc(C2(c3ccc(OCCO)cc3)c3ccccc3-c3ccccc32)cc1 ; COC(=O)OCCOc1ccc(C2(c3ccc(OCCOC(=O)OC)cc3)c3ccccc3-c3ccccc32)cc1 ; O=C(OCCOc1ccc(C2(c3ccc(OCCO)cc3)c3ccccc3-c3ccccc32)cc1)OCCOc1ccc(C2(c3ccc(OCCO)cc3)c3ccccc3-c3ccccc32)cc1 ; COC(=O)OCCOc1ccc(C2(c3ccc(OCCOC(=O)OCCOc4ccc(C5(c6ccc(OCCO)cc6)c6ccccc6-c6ccccc65)cc4)cc3)c3ccccc3-c3ccccc32)cc1]

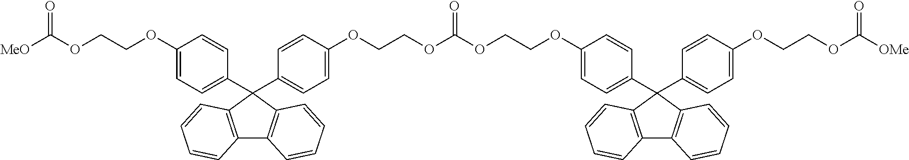 [SMILES: COC(=O)OCCOc1ccc(C2(c3ccc(OCCOC(=O)OCCOc4ccc(C5(c6ccc(OCCOC(=O)OC)cc6)c6ccccc6-c6ccccc65)cc4)cc3)c3ccccc3-c3ccccc32)cc1]

In the above description, an example of the reaction formula in a case where a hydroxy compound is used as the active hydrogen-containing compound has been described, but it is understood by those skilled in the art that a reaction product is generated according to the above reaction formula even in a case where the active hydrogen-containing compound is a compound other than the hydroxy compound (such as an amine compound or a thiol compound).

In the reaction of the present invention, the amount ratio between the active hydrogen-containing compound and the carbonate diester compound is not particularly limited as long as the target reaction proceeds. As the molar ratio, for example, [active hydrogen-containing compound]/[carbonate diester compound]=1/100 to 1/0.1 is preferable, [active hydrogen-containing compound]/[carbonate diester compound]=1/30 to 1/1 is more preferable, [active hydrogen-containing compound]/[carbonate diester compound]=1/20 to 1/2 is still more preferable, and [active hydrogen-containing compound]/[carbonate diester compound]=1/10 to 1/3 is also preferable.

The amount of the quaternary ammonium salt (catalyst) represented by General Formula (1) or (2) to be present in the reactor system in the reaction of the present invention is not particularly limited as long as it is an amount that catalyzes the target reaction. As the molar ratio, for example, [catalyst]/[carbonate diester compound]=1/10 to 1/10000 is preferable, [catalyst]/[carbonate diester compound]=1/10 to 1/2000 is more preferable, [catalyst]/[carbonate diester compound]=1/10 to 1/1000 is still more preferable, and [catalyst]/[carbonate diester compound]=1/12 to 1/500 is also preferable.

The reaction temperature of the reaction of the present invention depends on the type of the carbonate diester compound to be used, but is preferably in a range of 70 °C to 250 °C. This temperature is preferably a temperature equal to or higher than the boiling point of the alcohol, phenol, or the like, which is a by-product. Therefore, in a case where dimethyl carbonate is used as the diester carbonate, the by-product is methanol having a low boiling point, and thus the methanol as the by-product can be efficiently removed even in a case of a low-temperature reaction. In addition, by carrying out the reaction under reduced pressure, the removal of the by-product is facilitated. In consideration of reducing the environmental load while ensuring the reaction efficiency, the reaction temperature is more preferably 70 °C to 220 °C, more preferably 80 °C to 200 °C, and still more preferably 80 °C to 150 °C. In the reaction of the present invention, since the quaternary ammonium salt represented by General Formula (1) or (2) is used as the reaction catalyst, it is possible to appropriately apply a carbonate diester compound capable of removing the by-product at such a reaction temperature.

It is also preferable to control the reaction temperature by irradiating the reactor system with microwaves. By heating through microwave irradiation, the target carbonyl compound can be obtained with high efficiency even in a case where the reaction time is shortened (for example, the heating time is set to about ¼ or less of the heating time by an oil bath). The microwave may be a single mode or a multi-mode. In addition, the heating may be performed by an electric field or a magnetic field.

The reaction time of the reaction of the present invention is not particularly limited as long as the target reaction proceeds. For example, the reaction time can be set to 1 minute to 10 hours. In the batch reaction, the reaction time is preferably set to 5 minutes to 10 hours, and it can also be set to 20 minutes to 5 hours, and it is also preferably set to 30 minutes to 3 hours.

In addition, in a case where a flow reaction described later is adopted, the carbonyl compound can be obtained with high efficiency in a shorter reaction time. Therefore, in a case where the flow reaction is adopted, the reaction time is preferably set to 5 seconds to 30 minutes, and it can also be set to 10 seconds to 20 minutes, and it is also preferably set to 30 seconds to 10 minutes.

In a case where the reaction of the present invention is performed in a batch type, the active hydrogen-containing compound, the carbonate diester compound, and the quaternary ammonium salt represented by General Formula (1) or (2) are charged into a reaction container, and the reaction can be performed at the above-described reaction temperature. For example, the reaction can be performed while heating under reflux conditions in the air. The solvent can be appropriately used, but in a case where the raw material is in a liquid state under the reaction conditions, a solvent-free reactor system can also be adopted.

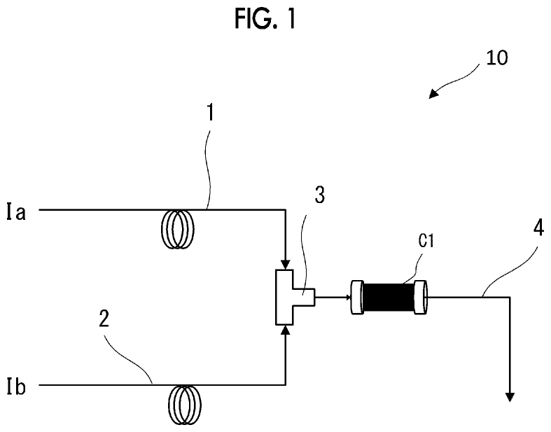

In a case where the reaction of the present invention is performed in a flow type, it is preferable that the quaternary ammonium salt represented by General Formula (1) or (2) is used as a solid-phase catalyst (solid catalyst). For example, by setting R¹ of General Formula (1) or (2) to a crosslinked polymer structure, the quaternary ammonium salt can be present in the reactor system as a solid. That is, R¹ can function as a carrier insoluble in the reaction solution. By filling a column with this insoluble catalyst and allowing a mixed solution of the active hydrogen-containing compound and the carbonate diester compound to flow through the column, the carbonyl group introduction reaction to the active hydrogen-containing compound proceeds while the mixed solution is flowing through the column. FIG. 1 schematically shows an example of a flow reaction system for performing the flow reaction. In a case where R¹ is a polymer structure insoluble in the reaction solution, the polymer structure does not necessarily have a crosslinking structure. Such an insoluble polymer structure can be appropriately designed.

In a flow reaction system (10) shown in FIG. 1, a liquid I containing the active hydrogen-containing compound is introduced from an introduction port (Ia) into a flow path (1), and a liquid II containing the carbonate diester compound is introduced from an introduction port (Ib) into a flow path (2). The introduction ports (Ia) and (Ib) are usually connected to a liquid feeding pump (not shown) such as a syringe pump or a diaphragm pump, and each liquid can be allowed to flow through each flow path at a desired flow rate by operating the pump.

The liquid I and the liquid II are combined at a confluence section (3), and the combined liquid is introduced into a column (C1) filled with a catalyst insoluble in the reaction solution, whereby the carbonyl group introduction reaction to the active hydrogen-containing compound can proceed in the column (C1). The reaction solution after the carbonyl group introduction reaction can be taken out through a pipe (4), and a target carbonyl compound can be obtained in the reaction solution.

The material, size, and the like of each flow path or the confluence section used in the flow reaction can be appropriately set in consideration of the target reaction scale.

In the reaction of the present invention, in a case where the active hydrogen-containing compound is, for example, a polyhydric alcohol compound, a polyvalent amine compound, or a polyvalent thiol compound, the polymerization reaction of the generated carbonyl compound can also proceed over time by extending the reaction time or the like, and an oligomer or a polymer can also be obtained. For example, in a case where a diol is used as the active hydrogen-containing compound, a polycarbonate compound can also be obtained by appropriately adjusting the reaction time, the reaction temperature, the reaction pressure, and the like. That is, the quaternary ammonium salt represented by General Formula (1) or (2) has excellent catalytic activity for the carbonyl group introduction reaction, and can also function as a polymerization catalyst for a compound into which a carbonyl group is introduced by this reaction. Therefore, according to the production method according to the embodiment of the present invention, a monomer carbonyl compound, a multimer (a dimer or higher oligomer or polymer) carbonyl compound, or a mixture thereof can be appropriately obtained depending on the purpose.

### <Solvent>

In a case where a solvent is used in the reaction of the present invention, a solvent capable of dissolving the active hydrogen-containing compound, the raw material such as the carbonate diester compound, and the carbonyl compound which is a product is preferable.

Examples of such a solvent include a halogen-containing solvent, a linear, branched, or cyclic ether solvent, and a hydrocarbon solvent.

Examples of the halogen-containing solvent include methylene chloride, chloroform, dichloroethane, carbon tetrachloride, chlorobenzene, and o-dichlorobenzene.

Examples of the ether solvent include tetrahydrofuran, dioxane, methyl tert-butyl ether, cyclopentyl methyl ether, ethylene glycol dibutyl ether, diethylene glycol dimethyl ether, diethylene glycol diethyl ether, diethylene glycol dibutyl ether, and derivatives thereof.

Examples of the hydrocarbon solvent include hexane, heptane, octane, cyclohexane, methylcyclohexane, benzene, toluene, xylene, mesitylene, decalin, tetralin, and derivatives thereof.

In addition, as the above-described solvent, a ketone-based solvent such as acetone, methyl ethyl ketone, diisobutyl ketone, cyclohexanone, or methyl isobutyl ketone, a nitrile-based solvent such as acetonitrile, a lactone-based solvent such as γ-butyrolactone, an ester-based solvent such as ethyl acetate or butyl acetate, an amide-based solvent such as dimethylacetamide or dimethylformamide, or the like can also be used.

The above-described solvent may be used alone or as a mixed solvent of two or more kinds thereof.

The present invention will be described in more detail based on Examples; however, the present invention should not be construed as being limited to these Examples except as defined in the present invention.

### EXAMPLES

### Example 1

A batch reaction was carried out as described below.

A 50 mL-sized eggplant-shaped flask was filled with 1.8 g (20 mmol) of the following C-1 as an active hydrogen-containing compound, 18.0 g (200 mmol) of dimethyl carbonate as a carbonate diester compound, and 0.348 g (1 mmol) of the following A-1 as a catalyst, and the mixture was reacted at 100 °C for 1 hour under reflux conditions in the air. The reaction solution was cooled to room temperature, and the components present in the reaction solution were analyzed by gas chromatography. Based on this analysis, the proportion (% by area) of the peak surface area of each of a compound in which only one of the two hydroxy groups contained in the above-described C-1 was converted into a methyl carbonate group (monosubstituted product), a compound in which both of the two hydroxy groups were converted into a methyl carbonate group (disubstituted product), and the above-described C-1 which was not reacted (unsubstituted product) with respect to the total peak surface area was measured, and the conversion rate of the hydroxy group contained in the above-described C-1 into a methyl carbonate group was calculated according to the following calculation expression. As a result, the conversion rate was 96%.

Conversion rate (%)=100×[(Area % of monosubstituted product)+(Area % of disubstituted product)×2]/[(Area % of unsubstituted product)×2+(Area % of monosubstituted product)×2+(Area % of disubstituted product)×2]

### Example 2

A batch reaction was carried out in the same manner as in Example 1, except that 23.6 g of diethyl carbonate was used as the carbonate diester compound instead of dimethyl carbonate.

The reaction solution was analyzed, and the conversion rate of the hydroxy group contained in the above-described C-1 into an ethyl carbonate group was calculated.

### Example 3

A batch reaction was carried out in the same manner as in Example 1, except that 34.8 g of dibutyl carbonate was used as the carbonate diester compound instead of dimethyl carbonate.

The reaction solution was analyzed, and the conversion rate of the hydroxy group contained in the above-described C-1 into a butyl carbonate group was calculated.

### Example 4

A batch reaction was carried out in the same manner as in Example 1, except that 42.8 g of diphenyl carbonate was used as the carbonate diester compound instead of dimethyl carbonate. The reaction solution was analyzed, and the conversion rate of the hydroxy group contained in the above-described C-1 into a phenyl carbonate group was calculated.

### Example 5

A batch reaction was carried out in the same manner as in Example 1, except that 8.76 g of the following C-2 was used as the active hydrogen-containing compound instead of the above-described C-1. The reaction solution was analyzed, and the conversion rate of the hydroxy group contained in the above-described C-2 into a methyl carbonate group was calculated.

### Example 6

A batch reaction was carried out in the same manner as in Example 5, except that 0.252 g of the following A-2 was used as the catalyst instead of the above-described A-1. The reaction solution was analyzed, and the conversion rate of the hydroxy group contained in the above-described C-2 into a methyl carbonate group was calculated.

### Example 7

A batch reaction was carried out in the same manner as in Example 5, except that 0.166 g of the following A-3 was used as the catalyst instead of the above-described A-1. The reaction solution was analyzed, and the conversion rate of the hydroxy group contained in the above-described C-2 into a methyl carbonate group was calculated.

### Example 8

A batch reaction was carried out in the same manner as in Example 5, except that 0.404 g of the following A-4 was used as the catalyst instead of the above-described A-1. The reaction solution was analyzed, and the conversion rate of the hydroxy group contained in the above-described C-2 into a methyl carbonate group was calculated.

### Example 9

A batch reaction was carried out in the same manner as in Example 5, except that 0.474 g of the following A-5 was used as the catalyst instead of the above-described A-1. The reaction solution was analyzed, and the conversion rate of the hydroxy group contained in the above-described C-2 into a methyl carbonate group was calculated.

### Example 10

A batch reaction was carried out in the same manner as in Example 5, except that 0.363 g of the following A-6 was used as the catalyst instead of the above-described A-1. The reaction solution was analyzed, and the conversion rate of the hydroxy group contained in the above-described C-2 into a methyl carbonate group was calculated.

### Example 11

A batch reaction was carried out in the same manner as in Example 5, except that 0.308 g of the following A-7 was used as the catalyst instead of the above-described A-1. The reaction solution was analyzed, and the conversion rate of the hydroxy group contained in the above-described C-2 into a methyl carbonate group was calculated.

### Example 12

A batch reaction was carried out in the same manner as in Example 5, except that 0.474 g of the following A-8 was used as the catalyst instead of the above-described A-1. The reaction solution was analyzed, and the conversion rate of the hydroxy group contained in the above-described C-2 into a methyl carbonate group was calculated.

### Example 13

A batch reaction was carried out in the same manner as in Example 5, except that 0.312 g of the following A-9 was used as the catalyst instead of the above-described A-1. The reaction solution was analyzed, and the conversion rate of the hydroxy group contained in the above-described C-2 into a methyl carbonate group was calculated.

### Example 14

A batch reaction was carried out in the same manner as in Example 5, except that 0.424 g of the following A-10 was used as the catalyst instead of the above-described A-1. The reaction solution was analyzed, and the conversion rate of the hydroxy group contained in the above-described C-2 into a methyl carbonate group was calculated.

### Example 15

A batch reaction was carried out in the same manner as in Example 5, except that 0.400 g of the following A-11 was used as the catalyst instead of the above-described A-1. The reaction solution was analyzed, and the conversion rate of the hydroxy group contained in the above-described C-2 into a methyl carbonate group was calculated. In Example 15, the catalyst A-11 was present as solid particles without being dissolved in the reaction solution, and the reaction solution was in a slurry form.

### Example 16

A flow reaction was carried out as follows using the flow reaction system shown in FIG. 1.

0.35 mL/min of dimethyl carbonate was fed from an introduction port (Ia) into a flow path (1) (a SUS tube, an inner diameter of 1.00 mm, a length of 500 mm, and a temperature of 30 °C), and 5.9 mL/min of the cyclopentyl methyl ether solution (0.033 g/mL) of the above-described C-2 was fed from an introduction port (Ib) into a flow path (2) (a SUS tube, an inner diameter of 1.00 mm, a length of 500 mm, and a temperature of 30 °C), and the two liquids were mixed in a confluence section (3) (a T-shaped mixer made of stainless steel (SUS) (an inner diameter of 0.5 mm and a temperature of 130 °C)). The mixed solution was allowed to flow through a SUS column (an inner diameter of 10 mm, a length of 100 mm, and a temperature of 130 °C) filled with the following A-11 as a solid catalyst, thereby causing the introduction reaction of the carbonyl group to proceed inside the column. The reaction solution that had passed through the column was analyzed by liquid chromatography (detection wavelength: 254 nm), and the conversion rate of the hydroxy group contained in the above-described C-2 into a methyl carbonate group was calculated in the same manner as described above. As a result, the conversion rate was 99%.

### Example 17

A flow reaction was carried out in the same manner as in Example 16, except that the following C-3 was used as the active hydrogen-containing compound instead of the above-described C-2, and the cyclopentyl methyl ether solution (0.017 g/mL) of the C-3 was fed into the flow path (2) at 5.9 mL/min. The reaction solution was analyzed, and the conversion rate of the hydroxy group contained in the above-described C-3 into a methyl carbonate group was calculated.

### Example 18

A flow reaction was carried out in the same manner as in Example 16, except that the following C-4 was used as the active hydrogen-containing compound instead of the above-described C-2, and the cyclopentyl methyl ether solution (0.020 g/mL) of the C-4 was fed into the flow path (2) at 5.9 mL/min. The reaction solution was analyzed, and the conversion rate of the hydroxy group contained in the above-described C-4 into a methyl carbonate group was calculated.

### Example 19

A flow reaction was carried out in the same manner as in Example 18, except that a cyclopentyl methyl ether solution (0.1 g/mL) of diphenyl carbonate was fed instead of dimethyl carbonate as the carbonate diester compound at 2.3 mL/min. The reaction solution was analyzed, and the conversion rate of the hydroxy group contained in the above-described C-4 into a phenyl carbonate group was calculated.

### Example 20

A flow reaction was carried out in the same manner as in Example 16, except that the following C-5 was used as the active hydrogen-containing compound instead of the above-described C-2, and the cyclopentyl methyl ether solution (0.020 g/mL) of the C-5 was fed into the flow path (2) at 5.9 mL/min. The reaction solution was analyzed, and the conversion rate of the hydroxy group contained in the above-described C-5 into a methyl carbonate group was calculated.

### Example 21

A flow reaction was carried out in the same manner as in Example 16, except that the following C-6 was used as the active hydrogen-containing compound instead of the above-described C-2, and the cyclopentyl methyl ether solution (0.023 g/mL) of the C-6 was fed into the flow path (2) at 5.9 mL/min. The reaction solution was analyzed, and the conversion rate of the hydroxy group contained in the above-described C-6 into a methyl carbonate group was calculated.

### Example 22

A batch reaction was carried out in the same manner as in Example 5, except that 0.34 g of the following A-12 was used as the catalyst instead of the above-described A-1. The reaction solution was analyzed, and the conversion rate of the hydroxy group contained in the above-described C-2 into a methyl carbonate group was calculated.

### Example 23

A batch reaction was carried out in the same manner as in Example 1, except that 4.56 g of the following C-3 was used as the active hydrogen-containing compound instead of the above-described C-1, and 0.412 g of the following A-13 was used as the catalyst instead of the above-described A-1. The reaction solution was analyzed, and the conversion rate of the hydroxy group contained in the above-described C-3 into a methyl carbonate group was calculated.

### Example 24

A batch reaction was carried out in the same manner as in Example 1, except that 5.36 g of the following C-4 was used as the active hydrogen-containing compound instead of the above-described C-1, and 0.484 g of the following A-14 was used as the catalyst instead of the above-described A-1. The reaction solution was analyzed, and the conversion rate of the hydroxy group contained in the above-described C-4 into a methyl carbonate group was calculated.

### Example 25

A batch reaction was carried out in the same manner as in Example 1, except that 6.20 g of the following C-6 was used as the active hydrogen-containing compound instead of the above-described C-1, and 0.393 g of the following A-15 was used as the catalyst instead of the above-described A-1. The reaction solution was analyzed, and the conversion rate of the hydroxy group contained in the above-described C-6 into a methyl carbonate group was calculated.

### Example 26

A batch reaction was carried out in the same manner as in Example 5, except that 0.440 g of the following A-16 was used as the catalyst instead of the above-described A-1. The reaction solution was analyzed, and the conversion rate of the hydroxy group contained in the above-described C-2 into a methyl carbonate group was calculated.

### Example 27

A batch reaction was carried out in the same manner as in Example 1, except that 3.00 g of the following C-7 was used as the active hydrogen-containing compound instead of the above-described C-1. The reaction solution was analyzed, and the conversion rate of the hydroxy group contained in the above-described C-7 into a methyl carbonate group was calculated. Since the above-described C-7 is a monoalcohol (monovalent phenol compound), the calculation expression of the conversion rate in Example 27 is as follows.

Conversion rate (%)=100×[(area % of substituted product)]/[(area % of unsubstituted product)+(area % of substituted product)]

### Example 28

A batch reaction was carried out in the same manner as in Example 27, except that 3.16 g of the following C-8 was used as the active hydrogen-containing compound instead of the above-described C-1. The reaction solution was analyzed, and the conversion rate of the hydroxy group contained in the above-described C-8 into a methyl carbonate group was calculated.

### Example 29

A batch reaction was carried out in the same manner as in Example 27, except that 2.14 g of the following C-9 was used as the active hydrogen-containing compound instead of the above-described C-1, the reaction solution was analyzed, and the conversion rate of the amino group contained in the above-described C-9 into a methyl carbonate group was calculated.

### Example 30

A batch reaction was carried out in the same manner as in Example 27, except that 1.98 g of the following C-10 was used as the active hydrogen-containing compound instead of the above-described C-1. The reaction solution was analyzed, and the conversion rate of the amino group contained in the above-described C-10 into a methyl carbonate group was calculated.

### Example 31

A batch reaction was carried out in the same manner as in Example 27, except that 3.48 g of the following C-11 was used as the active hydrogen-containing compound instead of the above-described C-1, the reaction solution was analyzed, and the conversion rate of the thiol group contained in the above-described C-11 into a methyl carbonate group was calculated.

### Example 32

A batch reaction was carried out in the same manner as in Example 1, except that 0.172 g of the following A-17 was used as the catalyst instead of the above-described A-1. The reaction solution was analyzed, and the conversion rate of the hydroxy group contained in the above-described C-1 into a methyl carbonate group was calculated.

### Example 33

A batch reaction was carried out in the same manner as in Example 1, except that 0.160 g of the following A-18 was used as the catalyst instead of the above-described A-1. The reaction solution was analyzed, and the conversion rate of the hydroxy group contained in the above-described C-1 into a methyl carbonate group was calculated.

### Comparative Example 1

A batch reaction was carried out in the same manner as in Example 5, except that 0.166 g of the following A-101 was used as the catalyst instead of the above-described A-1. The reaction solution was analyzed, and the conversion rate of the hydroxy group contained in the above-described C-2 into a methyl carbonate group was calculated.

### Comparative Example 2

A batch reaction was carried out in the same manner as in Example 5, except that 0.278 g of the following A-102 was used as the catalyst instead of the above-described A-1. The reaction solution was analyzed, and the conversion rate of the hydroxy group contained in the above-described C-2 into a methyl carbonate group was calculated.

### Comparative Example 3

A batch reaction was carried out in the same manner as in Example 5, except that 0.727 g of the following A-103 was used as the catalyst instead of the above-described A-1. The reaction solution was analyzed, and the conversion rate of the hydroxy group contained in the above-described C-2 into a methyl carbonate group was calculated.

### Comparative Example 4

A batch reaction was carried out in the same manner as in Example 5, except that 0.612 g of the following A-104 was used as the catalyst instead of the above-described A-1. The reaction solution was analyzed, and the conversion rate of the hydroxy group contained in the above-described C-2 into a methyl carbonate group was calculated.

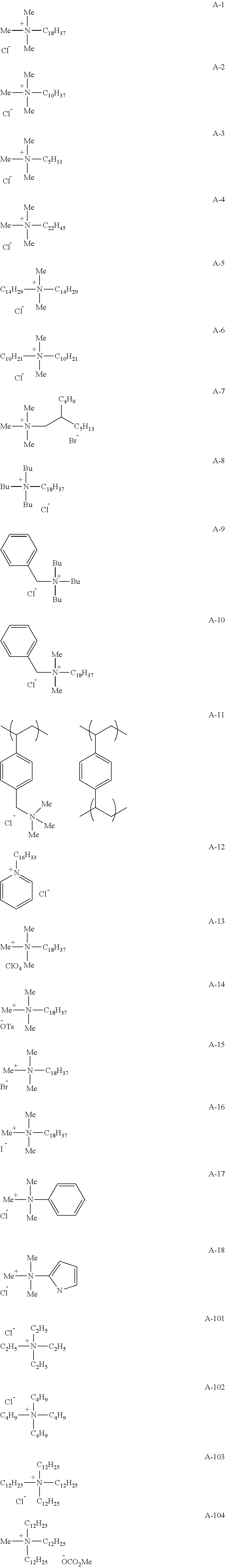

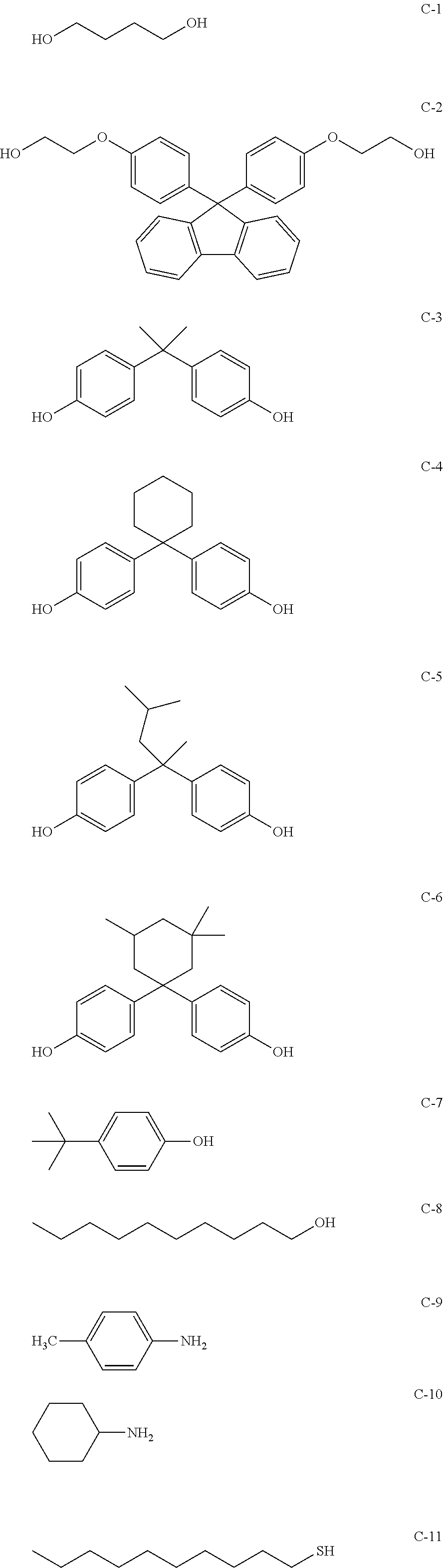

The results of each of Examples and Comparative Examples are summarized in the table below.

TABLE 1

| | | | Active | | |
|---|---|---|---|---|---|
| | | | hydrogen-containing | Reaction | Conversion |
| | Catalyst | Diester carbonate | compound | mode | rate (%) |
| Example 1 | A-1 | Dimethyl carbonate | C-1 | Batch type | 96 |
| Example 2 | A-1 | Diethyl carbonate | C-1 | Batch type | 90 |
| Example 3 | A-1 | Dibutyl carbonate | C-1 | Batch type | 79 |
| Example 4 | A-1 | Diphenyl carbonate | C-1 | Batch type | 98 |
| Example 5 | A-1 | Dimethyl carbonate | C-2 | Batch type | 98 |
| Example 6 | A-2 | Dimethyl carbonate | C-2 | Batch type | 93 |
| Example 7 | A-3 | Dimethyl carbonate | C-2 | Batch type | 85 |
| Example 8 | A-4 | Dimethyl carbonate | C-2 | Batch type | 95 |
| Example 9 | A-5 | Dimethyl carbonate | C-2 | Batch type | 93 |
| Example 10 | A-6 | Dimethyl carbonate | C-2 | Batch type | 88 |
| Example 11 | A-7 | Dimethyl carbonate | C-2 | Batch type | 93 |
| Example 12 | A-8 | Dimethyl carbonate | C-2 | Batch type | 65 |
| Example 13 | A-9 | Dimethyl carbonate | C-2 | Batch type | 61 |
| Example 14 | A-10 | Dimethyl carbonate | C-2 | Batch type | 69 |
| Example 15 | A-11 | Dimethyl carbonate | C-2 | Batch type | 98 |
| Example 16 | A-11 | Dimethyl carbonate | C-2 | Flow type | 99 |
| Example 17 | A-11 | Dimethyl carbonate | C-3 | Flow type | 89 |
| Example 18 | A-11 | Dimethyl carbonate | C-4 | Flow type | 89 |
| Example 19 | A-11 | Diphenyl carbonate | C-4 | Flow type | 94 |
| Example 20 | A-11 | Dimethyl carbonate | C-5 | Flow type | 91 |
| Example 21 | A-11 | Dimethyl carbonate | C-6 | Flow type | 90 |
| Example 22 | A-12 | Dimethyl carbonate | C-2 | Batch type | 65 |
| Example 23 | A-13 | Dimethyl carbonate | C-3 | Batch type | 68 |
| Example 24 | A-14 | Dimethyl carbonate | C-4 | Batch type | 73 |
| Example 25 | A-15 | Dimethyl carbonate | C-6 | Batch type | 94 |
| Example 26 | A-16 | Dimethyl carbonate | C-2 | Batch type | 92 |
| Example 27 | A-1 | Dimethyl carbonate | C-7 | Batch type | 97 |
| Example 28 | A-1 | Dimethyl carbonate | C-8 | Batch type | 97 |
| Example 29 | A-1 | Dimethyl carbonate | C-9 | Batch type | 98 |
| Example 30 | A-1 | Dimethyl carbonate | C-10 | Batch type | 96 |
| Example 31 | A-1 | Dimethyl carbonate | C-11 | Batch type | 94 |
| Example 32 | A-17 | Dimethyl carbonate | C-1 | Batch type | 74 |
| Example 33 | A-18 | Dimethyl carbonate | C-1 | Batch type | 68 |
| Comparative | A-101 | Dimethyl carbonate | C-2 | Batch type | 37 |
| Example 1 | | | | | |
| Comparative | A-102 | Dimethyl carbonate | C-2 | Batch type | 42 |
| Example 2 | | | | | |
| Comparative | A-103 | Dimethyl carbonate | C-2 | Batch type | 38 |
| Example 3 | | | | | |
| Comparative | A-104 | Dimethyl carbonate | C-2 | Batch type | 34 |
| Example 4 | | | | | |

From the above results, it was found that, even in a case where a quaternary ammonium salt was used as the catalyst, in a case where the chemical structure thereof did not satisfy the regulations of the present invention, the introduction efficiency of the carbonyl group into the active hydrogen-containing compound was deteriorated (Comparative Examples 1 to 4).

On the other hand, it was found that, in a case where the active hydrogen-containing compound and the carbonate diester compound were reacted with each other in the presence of the quaternary ammonium salt represented by General Formula (1) or (2), the introduction efficiency of the carbonyl group into the active hydrogen-containing compound was significantly increased regardless of the batch type or flow type reaction mode (Examples 1 to 33).

### Example 34

A glass container (volume: 5 mL) dedicated to a microwave synthesizer was filled with 0.36 g of the above-described C-1 as the active hydrogen-containing compound, 3.6 g of dimethyl carbonate as the carbonate diester compound, and 0.070 g of the above-described A-1 as the catalyst, and the container was sealed with a septum. Subsequently, using a microwave reaction device (Initiator+ manufactured by Biotage, LLC, maximum output: 400 W), the inside of the glass container was irradiated with microwaves while being stirred, the liquid in the glass container was heated to 130 °C, and the reaction was carried out in this state for 15 minutes. The reaction solution was analyzed, and the conversion rate of the hydroxy group contained in the above-described C-1 to the methyl carbonate group was calculated, and the conversion rate was 95%. From this result, it was found that, by adopting microwave heating, the introduction of the carbonyl group into the active hydrogen-containing compound can be carried out with high efficiency in a short time.

### Example 35

An eggplant-shaped flask having a size of 50 mL was filled with 1.8 g (20 mmol) of the above-described C-1 as the active hydrogen-containing compound, 18.0 g (200 mmol) of dimethyl carbonate as the carbonate diester compound, and 0.348 g (1 mmol) of the above-described A-1 as the catalyst, and the mixture was reacted at 100 °C for 1 hour under reflux conditions under atmospheric pressure. The reaction was carried out at 110 °C for 1 hour under atmospheric pressure, and then at 150 °C for 1.5 hours under reduced pressure (20 Torr). The reaction solution was cooled to room temperature, and as a result of analysis by gel permeation chromatography, a polymer having a weight-average molecular weight of 5,100 was produced. This result shows that, by appropriately controlling the reaction conditions, it is possible to obtain a low-molecular-weight carbonyl compound (monomer) or a polymer (a prepolymer or a polymer) of this low-molecular-weight carbonyl compound.

The present invention has been described using the embodiments. However, unless specified otherwise, any of the details of the above description is not intended to limit the present invention and can be construed in a broad sense within a range not departing from the concept and scope of the present invention disclosed in the accompanying claims.

### EXPLANATION OF REFERENCES

10: flow reaction system
Ia, Ib: introduction port
1, 2: flow path
3: confluence section
4: pipe
C1: solid-phase catalyst (column filled with solid catalyst)

What is claimed is:

1. A method for producing a carbonyl compound, the method comprising:
reacting an active hydrogen-containing compound having, in a molecule, an active hydrogen-containing group formed by bonding a hydrogen atom to an atom selected from an oxygen atom, a nitrogen atom, and a sulfur atom, with a carbonate diester compound in a presence of a quaternary ammonium salt represented by General Formula (1) or (2), thereby introducing a carbonyl group into the active hydrogen-containing compound, 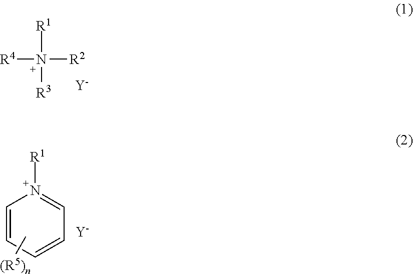
in General Formulae (1) and (2), R¹ represents an organic group having a formula weight of 65 or more, R² to R⁴ each represent an alkyl group, and at least two of R², R³, or R⁴ are alkyl groups having 6 or fewer carbon atoms, R⁵ represents an alkyl group having 1 to 20 carbon atoms, and n is an integer of 0 to 2, and Y⁻ represents a counterion.

2. The method for producing a carbonyl compound according to claim 1,
wherein in General Formulae (1) and (2), R¹ represents an alkyl group having 5 to 30 carbon atoms or a polymer structure.

3. The method for producing a carbonyl compound according to claim 2,
wherein in General Formulae (1) and (2), R¹ is a polymer structure having a crosslinking structure.

4. The method for producing a carbonyl compound according to claim 3,
wherein the quaternary ammonium salt is present as a solid in a reactor system of the reaction.

5. The method for producing a carbonyl compound according to claim 4,
wherein the reaction is performed in a flow reactor system.

6. The method for producing a carbonyl compound according to claim 5, the method further comprising:
causing a liquid containing the active hydrogen-containing compound and the carbonate diester compound to flow through an inside of a column filled with the quaternary ammonium salt.

7. The method for producing a carbonyl compound according to claim 1,
wherein in General Formula (1), at least two of R², R³, or R⁴ are methyl.

8. The method for producing a carbonyl compound according to claim 1,
wherein in General Formulae (1) and (2), Y⁻ is selected from a halide anion, a sulfonate anion, a phosphate anion, a phosphonate anion, a perchlorate anion, and a fluorophosphate anion.

9. The method for producing a carbonyl compound according to claim 1,
wherein the carbonate diester compound is a dialkyl carbonate compound.

10. The method for producing a carbonyl compound according to claim 9,
wherein the carbonate diester compound is dimethyl carbonate.

11. The method for producing a carbonyl compound according to claim 1,
wherein the active hydrogen-containing compound is selected from a polyhydric alcohol compound, a polyvalent amine compound, and a polyvalent thiol compound.

12. The method for producing a carbonyl compound according to claim 11,
wherein the carbonyl compound obtained by the production method is a monomer and/or a multimer.

13. The method for producing a carbonyl compound according to claim 1, the method further comprising:
irradiating a reactor system of the reaction with a microwave to heat the reactor system.
